(19) United States
(12) Patent Application Publication
(10) Pub. No.: US 2014/0330185 A1
(43) Pub. Date: Nov. 6, 2014

(54) Orthopedic Device For Use With An Orthopedic Cast

(71) Applicants: Mohammad Taslim, Needham, MA (US); Benjamin Hall, Smithtown, NY (US); Zubin Joy, Niskayuna, NY (US); Bhavik Raval, Totowa, NJ (US); Justin Tufariello, Ridgefiled, CT (US); Lawrence Wong, Brooklyn, NY (US); Melissa Davis, Tiverton, RI (US)

(72) Inventors: Mohammad Taslim, Needham, MA (US); Benjamin Hall, Smithtown, NY (US); Zubin Joy, Niskayuna, NY (US); Bhavik Raval, Totowa, NJ (US); Justin Tufariello, Ridgefiled, CT (US); Lawrence Wong, Brooklyn, NY (US); Melissa Davis, Tiverton, RI (US)

(21) Appl. No.: 14/270,009

(22) Filed: May 5, 2014

Related U.S. Application Data
(60) Provisional application No. 61/819,390, filed on May 3, 2013.

(51) Int. Cl. A61F 5/058

(52) U.S. Cl. CPC A61F 5/05816
USPC 602/13

(57) ABSTRACT

The present invention is generally directed to an orthopedic device for use with an orthopedic cast, which can alleviate the itching sensation that can develop when a patient wears a cast, among other advantages, such as cooling the skin below the cast, reducing the moisture in the region between the skin and the cast, as well as in some cases reducing the risk of infection. As discussed in more detail below, the orthopedic device can include a sleeve that can be mounted onto a body part, e.g., a broken limb, between the skin and a surgical cast. The sleeve is sufficiently flexible so as to substantially conform to the contour of the body part. The sleeve includes a plurality of internal channels for receiving a gas from an external source, e.g., a canister of pressurized gas, and for delivering at least a portion of the received gas via a plurality of openings onto the skin below the cast. The sleeve can include a two opposed layers between which the internal channels are formed.

### RELATED APPLICATION

The present application claims priority to a provisional application having Application No. 61/819390, filed on May 3, 2013, which is herein incorporated by reference in its entirety.

### BACKGROUND

The present invention is generally directed to orthopedic devices and systems for use with orthopedic casts, and more particularly, to sleeves that can be mounted onto a subject's body part, e.g., a limb, to deliver a gas flow onto the skin below an orthopedic cast. The gas flow can ameliorate a patient's discomfort, such as itching, and can reduce the moisture level below the cast, and in some cases reduce the risk of infection.

One of the most common orthopedic problems is broken bones. Orthopedic surgeons cast broken limbs to ensure their proper recovery. In one common casting technique, a cotton sleeve is slid over the injured area and a cotton gauze is wrapped over the sleeve to provide a certain degree of rigidity. Subsequently, a casting material made of plaster or fiberglass is applied as the final layer of the cast. A patient wearing a surgical cast can develop an itch in the skin below the cast. Such itching may develop, e.g., as a response to moisture trapped below the cast, the lack of airflow and/or the build-up of body salts and dead skin. In addition, the trapped body sweat under the cast may cause unpleasant odors. As a surgical cast is worn typically for a few weeks (e.g., four to twelve weeks), the build-up of salts and dead skin under the cast over this period may lead to infection.

Accordingly, there is a need for orthopedic devices and systems, as well as methods for their use, that can address the above problems associated with surgical casts.

### SUMMARY

In one aspect, an orthopedic device for use with an orthopedic cast is disclosed, which comprises a sleeve having opposed top and bottom layers and adapted for mounting onto a subject's body part such that the bottom layer is disposed proximate to, or in contact, with the skin. The opposed top and bottom layers are connected to one another so as to form an input channel, a distribution channel and at least one gas-delivery channel in a space therebetween. The input channel comprises an opening (an inlet) at a proximal end thereof for receiving gas from an external source. The input channel is fluidly coupled at its distal end to the distribution channel so to deliver at least a portion of the received gas to the distribution channel. The distribution channel is in turn configured to deliver at least a portion of the gas to each of said plurality of gas-delivery channels. A plurality of openings are disposed in the bottom layer to allow the gas to exit from at least of one of said channels out of the sleeve, e.g., onto the subject's skin and/or a region proximate to the skin below an orthopedic cast. In some embodiments, the channels can have a width in a range of about 0.3 inches (about 7.6 mm) to about 0.5 inches (about 7.6 mm).

Each of the channels includes a top wall and a bottom wall, where the top wall comprises a portion of the top layer of the sleeve and the bottom wall comprises a portion of the bottom layer of the sleeve. In some embodiments, the openings are disposed along a bottom wall of at least one of said channels, and in some cases, along the bottom walls of all of the channels. While in some embodiments the openings are spaced uniformly relative to one another, in others, the openings are randomly distributed relative to one another. The openings can have a variety of different shapes and sizes. For example, the openings can have a circular, an elliptical, a square, or rectangular, or an irregular, shape. By way of example, in some embodiments, the openings can be circular with a diameter in a range of about 0.8 mm to about 1.6 mm.

In some embodiments, each of the top and bottom layer can have a thickness in a range of about 50 micrometers (microns) to about 250 microns. The top and the bottom layers can be formed of any suitable biocompatible polymeric material. By way of example, in some embodiments, the top and bottom layers are formed of polyethylene.

In some embodiments, the bottom layer has a corrugated outer surface. In some such embodiments, the corrugated surface includes a plurality of raised portions forming a plurality of grooves therebetween. When the sleeve is mounted onto a body part, the grooves can form cavities that can facilitate contact between the gas exiting the sleeve and the skin. In some embodiments, the openings are formed along the grooves.

In some embodiments, the sleeve includes multiple gas-delivery channels, each of which is configured to be in fluid communication with the distribution channel. In some embodiments, the gas-delivery channels are substantially parallel to one another and are substantially orthogonal to the distribution channel.

A gas-delivery channel can extend from a proximal end, which is in fluid communication with the distribution channel, to a distal end. In some embodiments, one or more of the gas-delivery channels can exhibit a tapered shape. By way of example, a tapered gas-delivery channel can exhibit a width that decreases as a function of increasing distance from its proximal end. In other words, in some embodiments, one or more of the gas delivery channels can exhibit a progressive narrowing as a function of increasing distance from the distribution channel. In some other embodiments, one or more of the tapered channels can exhibit a width that increases as a function of increasing distance from its proximal end. In other words, in some embodiments, one or more gas channels can exhibit a progressively increasing width as a function of increasing distance from the distribution channels. In some embodiments, a tapered channel can be characterized by a taper angle in a range of about 10 degrees to about 30 degrees.

In some embodiments, a porous drug-delivery element can be coupled to an orthopedic device according to the present teachings to deliver a therapeutic agent onto the skin below an orthopedic cast. In some such embodiments, the gas flow provided by the sleeve can facilitate the transfer of a therapeutic agent from the drug-delivery element onto the skin. By way of example, the orthopedic device can include a porous drug-delivery element disposed in said at least one of the channels. By way of example, the porous drug-delivery element can be disposed in one or more of the gas-delivery channels. In some embodiments, the porous drug-delivery element comprises a vinyl polymer, e.g., a vinyl acetate polymer.

In some embodiments, an external gas source coupled to the sleeve comprises a container of compressed gas. Any suitable gas can be employed in the practice of the present teachings. By way of example, the gas can be any of CO₂, N₂, air (e.g., dry air) and/or argon. In some embodiments, the gas can be a mixture of two of more gases. The gas container can include a regulator for adjusting the gas pressure and a trigger mechanism for adjusting the flow rate of the gas exiting the container. In some embodiments, the container can be coupled to the sleeve via a tube that can be connected to gas fitting, e.g., a quick connect, coupled to the opening of the input channel of the sleeve.

In a related aspect, a sleeve for use with an orthopedic cast is disclosed, which comprises a top layer and a bottom layer connected to one another to provide an enclosure therebetween. A plurality of channels are disposed in said enclosure, where the channels comprise an input channel having an inlet port for receiving a gas from a source, a distribution channel fluidly coupled to said input channel to receive at least a portion of the gas flowing in the input channel, and one or more gas-delivery channels fluidly coupled to said distribution channel to receive at least a portion of the gas flowing in the distribution channel. A plurality of openings are disposed in said bottom layer to deliver at least a portion of the gas flowing in at least one of said channels to an external environment.

In some embodiments, the openings are disposed along at least one of said channels. In some embodiments, the distribution channel is substantially orthogonal to the input channel and the gas-delivery channels are substantially perpendicular to the distribution channel.

In yet another related aspect, an orthopedic device for use with an orthopedic cast is disclosed, which comprises a sleeve adapted for mounting onto a body part, said sleeve comprising at least one layer configured to form an internal enclosure. A plurality of channels are disposed in said enclosure, where the channels comprise an input channel having an inlet for receiving gas from a source, a distribution channel fluidly coupled to said input channel and one or more gas-delivery channels fluidly coupled to any of said distribution channel and said input channel.

In some embodiments, the distribution channel is substantially orthogonal to said input channel, and the one or more gas-delivery channels are substantially orthogonal to said distribution channel.

In some embodiments, an orthopedic device for use with an orthopedic cast may include a sleeve formed from at least two substantially circular cuffs configured to encircle a portion of a limb covered by the orthopedic cast. The cuffs may include openings on facing surfaces thereof and a plurality of channels may be configured to connect corresponding openings on the at least two cuffs, thereby connecting the at least two cuffs in fluid communication. At least one cuff may include a port configured to receive a gas from an external source to introduce gas into the sleeve. At least a portion of the plurality of channels may include perforations (openings) to allow the passage of gas onto the skin.

In some embodiments, an orthopedic device for use with a surgical cast may include a sleeve (a “bladder sleeve”) adapted for placement between skin and a casting material of the surgical cast. The sleeve may include a bladder configured to encircle a portion of a limb. The bladder may include a plurality of channels arranged substantially parallel to one another. A port may be coupled to the bladder to introduce a gas from an external source into the plurality of channels. A plurality of perforations (openings) may be disposed on each of the plurality of channels to allow passage of the gas from the plurality of channels onto the skin.

In a related aspect, an orthopedic device for use with an orthopedic cast is disclosed, which includes a sleeve having a top layer and bottom layer, where the layers are joined together to form an enclosure therebetween. The sleeve is adapted for mounting onto a body part, e.g., a limb. The sleeve further includes an inlet through which a gas from an external gas source can be introduced into the enclosure between the top and the bottom layers. The bottom layer of the sleeve is formed of a porous material, e.g., porous polyurethane, to allow the gas introduced into the enclosure, or at least a portion thereof, to exit the sleeve. When sleeve is mounted between a subject's skin and an orthopedic cast, the gas exiting the sleeve can flow over the skin. The flow of the gas over the skin can provide a number of advantages, e.g., alleviating an itching sensation, cooling the skin, reducing the moisture in a space between the skin and the cast, and in some cases reducing the risk of infection.

Further understanding of various aspects of the invention can be obtained by reference to the following detailed description in conjunction with the associated drawings, which are described briefly below.

### BRIEF DESCRIPTION OF THE DRAWINGS

FIG. 1A schematically depicts an orthopedic device in accordance with an embodiment of the present teachings, which comprises a sleeve adapted for mounting onto a body part,

FIG. 1B is a schematic view of bottom layer of the orthopedic device of the present teachings, where the bottom layer is configured to be facing the skin upon mounting the sleeve onto a body part,

FIG. 1C is a schematic view of a top layer of the orthopedic device shown in FIG. 1A, where the top layer is configured to be facing a cast upon mounting the sleeve onto a body part,

FIG. 1D is a schematic view of the bottom layer of the sleeve depicted in FIG. 1A, where the arrows show the flow of gas through internal channels of the sleeve,

FIG. 2 schematically shows an outside surface of the bottom layer of a sleeve according to the present teachings, which includes a corrugated surface,

FIG. 3A schematically shows a view of a plurality of channels of a sleeve according to an embodiment of the present teachings in which one or more of the channels are tapered,

FIG. 3B schematically shows a tapered channel in which the taper of the channel is characterized by a taper angle (α),

FIG. 3C schematically shows a sleeve according to an embodiment of the invention, which includes one or more of gas delivery channels exhibiting a progressively increasing width from the distribution channel to an edge of the sleeve,

FIG. 4A shows schematically a view of a portion of sleeve according to the present teachings in which one or more drug-delivery elements are disposed in selected channels of the sleeve,

FIG. 4B shows schematically a view of a sleeve according to the present teachings, which includes one or more tapered gas-delivery channels,

FIG. 4C shows schematically the sleeve of FIG. 4B and its connection to a gas container,

FIG. 5A is a partial schematic view of a sleeve according to the present teachings depicting a quick connect coupled to the inlet of an input channel of the sleeve,

FIG. 5B is partial schematic view of a sleeve according to the present teachings depicting the coupling of the sleeve to a gas container,

FIG. 6A shows a gas container suitable for use in the present teachings,

FIG. 6B shows coupling of an upper portion of a housing to the gas container,

FIG. 6C shows the coupling of an upper and a lower portion of a housing to the gas container,

FIG. 6D shows exemplary dimensions of the housing shown in FIGS. 6B and 6C,

FIG. 7A shows an upper portion a housing according to an embodiment for coupling to a gas container, where the upper portion includes an opening for accessing a nozzle of the gas container,

FIG. 7B shows a housing, which houses a gas container, and a sleeve according to an embodiment of the present teachings coupled to the gas container,

FIG. 7C shows a sleeve according to another embodiment of the present teachings, which includes a porous bottom layer through which a gas can be delivered to the skin below an orthopedic cast,

FIG. 8 shows a prototype of a flow sleeve according to the present teachings,

FIGS. 9A-9C show one exemplary way of mounting the prototype of FIG. 8 to a forearm,

FIGS. 10A-10C schematically depict an orthopedic device in accordance with an embodiment of the present teachings, which comprises a sleeve adapted for mounting to a body part,

FIG. 11A schematically depicts a cuff component of an orthopedic device in accordance with an embodiment of the present teachings,

FIG. 11B schematically depicts an orthopedic device in accordance with an embodiment of the present teachings, and

FIG. 12 schematically depicts an orthopedic device in accordance with another embodiment of the present teachings.

### DETAILED DESCRIPTION

The present invention is generally directed to an orthopedic device for use with an orthopedic cast, which can alleviate the itching sensation that can develop when a patient wears a cast, among other advantages, such as cooling the skin below the cast, reducing the moisture in the region between the skin and the cast, as well as in some cases reducing the risk of infection. As discussed in more detail below, the orthopedic device can include a sleeve that can be mounted onto a body part, e.g., a broken limb, between the skin and an orthopedic cast. The sleeve is sufficiently flexible so as to substantially conform to the contour of the body part. The sleeve includes a plurality of internal channels for receiving a gas from an external source, e.g., a canister of pressurized gas, and for delivering at least a portion of the received gas via a plurality of openings onto the skin below the cast. The sleeve can include a two opposed layers between which the internal channels are formed.

The term “about” as used herein indicates a variation of at most 5%. The term “substantially” as used herein indicates a deviation of less than 5%. The term “fluidly coupled” indicates that two components, e.g., two channels of a sleeve according to the present teachings, can exchange a fluid, e.g., a gas, therebetween, e.g., via a flow from one component to another.

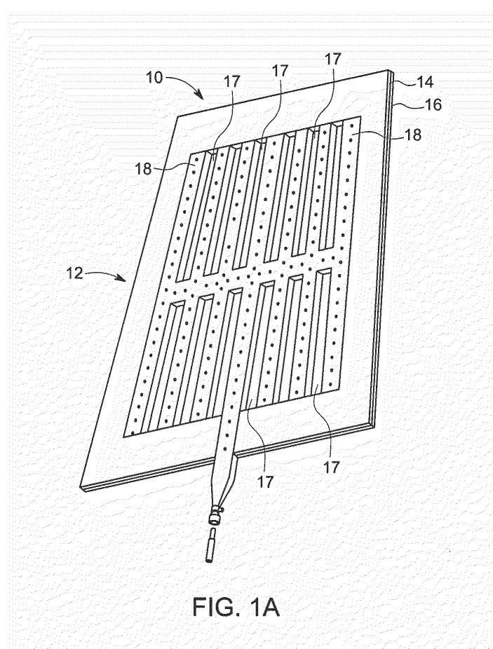

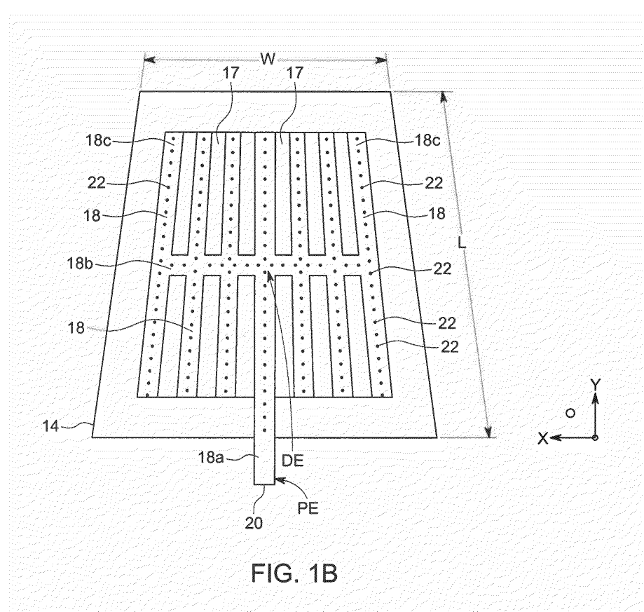

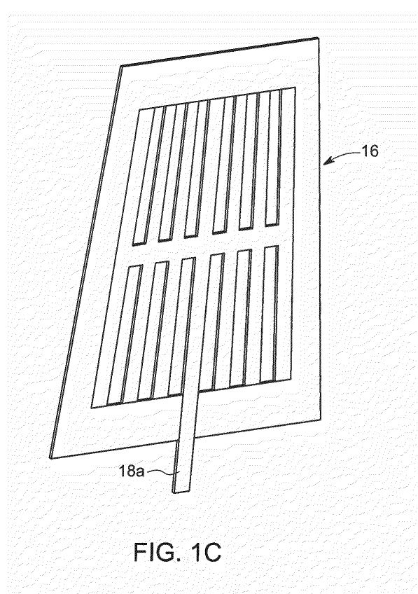

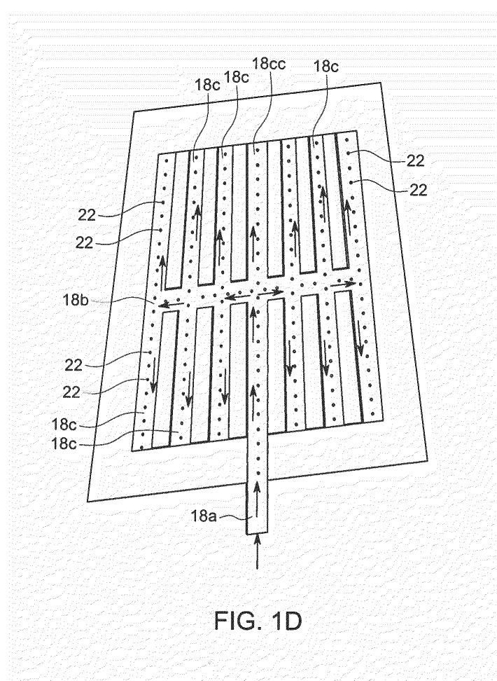

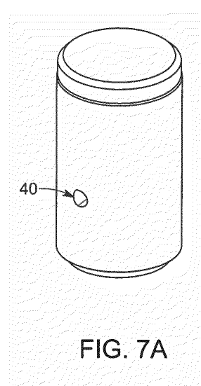

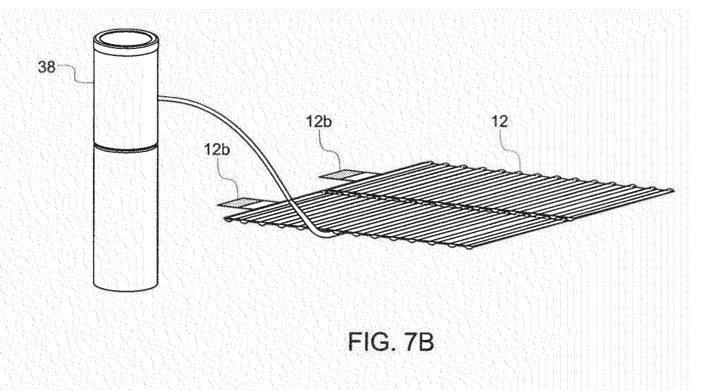

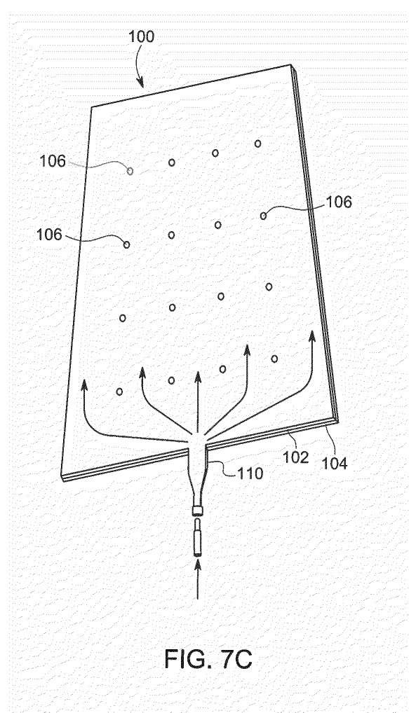

With reference to FIGS. 1A, 1B, 1C, an orthopedic device 10 according to an embodiment of the present teachings includes a sleeve 12 (herein also referred to as a “flow sleeve”), which is configured to be mounted onto the exterior of a subject's body part, e.g., a limb, between the skin and an orthopedic cast. In this embodiment, the sleeve 12 is formed of a conformable biocompatible polymeric material, e.g., polyethylene polymer, and is sufficiently flexible to conform substantially to the contour of a body part, e.g., flexible enough to be wrapped around a subject's limb, e.g., a broken forearm. The sleeve is preferably sized so as to fit snugly around the exterior of a body part onto which the sleeve is mounted. In some embodiments, the sleeve 12 can include a fastener, e.g., hook-and-loop mechanism, (not shown in this figure) for fixating it in place (such a fastener is shown in the embodiment depicted in FIG. 7B, which is discussed below).

The sleeve 12 can have a variety of different sizes, e.g., based on a particular application. By way of example, in some embodiments, the sleeve 10 can have length (L) in a range of about 12 inches (about 30.5 cm) to about 20 inches (about 50.8 cm) and a width in a range of about 8 inches (about 20.3 cm) to about 12 inches (about 30.5 cm). As discussed in more detail below, the sleeve 10 is wrapped around a limb, e.g., a broken forearm, along its width dimension with the length dimension corresponding to the extension of the sleeve along the limb.

In this embodiment, the sleeve 12 includes a bottom layer 14 and an opposed top layer 16. The terms “top” and “bottom” are used herein to distinguish between a layer that is adapted to face the skin (e.g., to be proximate to and/or in contact with the skin) when the sleeve is mounted onto a body part (herein referred to as the “bottom layer”) and the opposed layer (herein referred to as the “top layer”), which faces a casting material (e.g., it is in contact with the casting material) when the sleeve is mounted to a body part. As in many embodiments, the sleeve can have a substantially cylindrical shape once it is mounted onto a body part, the bottom layer is also herein referred to in some cases as the “front layer” and the top layer as the “back layer.” It should be understood that the terms “top,” “bottom,” “front,” and “back” are only employed for ease of illustration of various features of an orthopedic device according to the present teachings, and not to limit the scope of the present invention.

With continued reference to FIGS. 1A, 1B, and 1C, the top layer 16 is connected to the bottom layer 14 so as to form a plurality of channels (herein also referred to as passageways) 18 in the internal space between the two layers such that portions of the top layer 14 form top walls of the channels and portions of the bottom layers 16 form bottom walls of the channels.

With continued reference to FIGS. 1A, 1B, and 1C as well as FIG. 1D, in this embodiment, the channels 18 include an input channel 18a, a gas distribution channel 18b and a plurality of gas-delivery channels 18c. The input channel 18a extends from a proximal end (PE) to a distal end (DE). The proximal end of the input channel provides an inlet 20 for receiving a gas from an external source, e.g., a container of compressed gas, as discussed in more detail below. The distal end of the input channel 18a is fluidly connected to the distribution channel 18b (herein also referred to as the “gas distribution channel”) to allow the flow of an input gas (or at least a portion thereof) from the input channel into the distribution channel. The distribution channel 18b extends along a width of the sleeve 12 and is fluidly coupled to the plurality of gas-delivery channels 18c to distribute the gas received from the input channel (or at least a portion thereof) among the gas-delivery channels. In this embodiment, a plurality of gaps 17 (e.g., cut-out portions) separate adjacent gas-delivery channels from one another. In other embodiments, such gaps may not be present. The arrows in FIG. 1D schematically depict the paths of gas flow through the channels of the sleeve. One or more of the gas-delivery channels (e.g., channel 18cc) can receive a gas flow not only from the distribution channel 18b, but also directly from the input channel 18a. Although this embodiment includes one input channel and one distribution channel, in other embodiments multiple input channels and distribution channels can be provided.

The sleeve 12 further includes a plurality of openings 22 formed in the bottom layer 14 and distributed along the lower walls of the internal channels (i.e., the input channel 18a, the distribution channel 18b and the gas-delivery channels 18c). The openings 22 allow the exit of the gas from the channels onto the skin of a body part, e.g., a limb, onto which the sleeve is mounted. Hence, in this embodiment, the input channel not only delivers a portion of the gas received from an external source to the distribution channel, but it also allows a portion of the received gas to escape through some of the openings 22, which are provided along its bottom wall, once the sleeve is mounted onto a body part. Similarly, in this embodiment, the distribution channel 18b not only distributes a portion of the gas received from the input channel to the gas-delivery channels 18c but it also allows a portion of the gas to escape the sleeve through the openings provided in its bottom wall onto the skin. In other embodiments, the openings can be provided along a subset of the channels. For example, in some embodiments, the openings are distributed only along the gas-delivery channels, and not along the input and the distribution channels.

In this embodiment, the openings 22 are disposed along the channels in a regular arrangement such that adjacent openings are spaced from one another substantially uniformly. In other embodiments, the openings 22 can be randomly distributed along the bottom walls of the channels.

The openings 22 can have a variety of different shapes and sizes. For example, the openings can be circular, elliptical, square, or any other suitable shape, including an irregular shape. In some embodiments, the openings are substantially circular with a diameter in a range of about 0.8 millimeters (mm) to about 1.6 mm (corresponding to a range of about 1/32 inches to about 1/16 inches). In some embodiments, the openings can have different sizes. For example, the openings along the input channel and the distribution channel can be smaller than the openings along the gas-delivery channels.

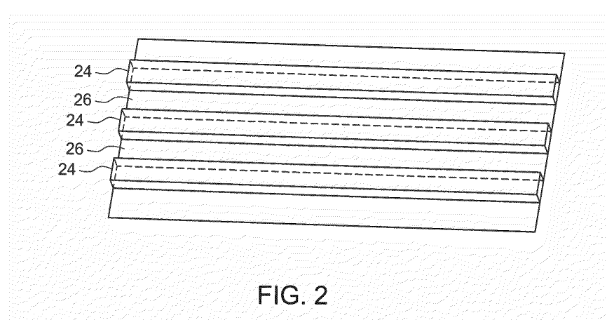

As shown schematically in FIG. 2, in some embodiments, the bottom layer of the sleeve can be formed as a contiguous layer having a corrugated outer surface characterized by a plurality of raised portions 24 separated by a plurality of grooves 26 therebetween. The grooves 26 can provide a plurality of cavities between the skin and the sleeve when the sleeve is mounted onto a body part, where the cavities can facilitate contact between the gas exiting the sleeve and the skin. In some embodiments, the grooves 26 are formed along one or more the channels, e.g., along the gas-delivery channels, to facilitate the delivery of the gas onto the skin.

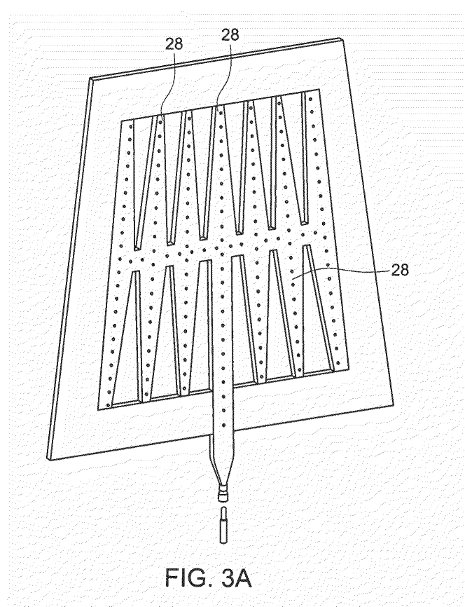

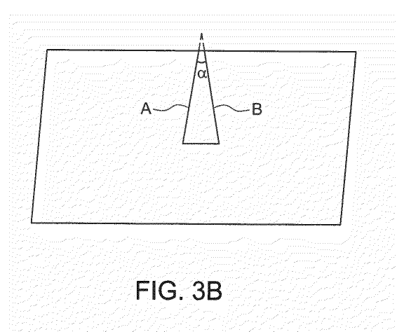

In some embodiments, at least one, and preferably all, of the gas-delivery channels can be tapered such that the channel's width increases from proximal end of the channel, which is coupled to the gas-distribution channel, to a distal end thereof, which can be proximate to an edge of the sleeve. In other words, the channel can be progressively narrowed as a function of increasing distance from the distribution channel. By way of illustration, FIG. 3A schematically depicts such an embodiment having a plurality of tapered gas-delivery channels 28. As shown schematically in FIG. 3B, the taper of the channels can be characterized by a taper angle (α), which is defined by putative extensions of the lines A and B, which delimit the channel's width (a dimension perpendicular the channels length), to a point at which they intersect (for each of illustration, FIG. 3B shows only one tapered channel). In some embodiments, the angle (α) can be in a range of about 10 to about 30 degrees. In some embodiments, the tapered channels can facilitate a more uniform distribution of the gas onto a subject's skin when the sleeve is mounted to a body part, e.g., a broken limb. For example, as the gas travels from a proximal end of the tapered channels 28 to their distal end, the velocity of the gas can increase due to the narrowing of the channels as the gas flows from the proximal end of the channel toward the distal end thereof, thereby facilitating the delivery of the gas to the downstream openings, e.g., the opening close to distal end of the channels. For example, as the gas moves through a gas-delivery channel, some of the gas leaves the channel through the holes disposed at its bottom. The taper of a channel can help delivering some of the gas to the openings at the downstream portion of the channel. In some embodiments, the sizes of the openings in a tapered channel can be non-uniform. For example, the openings proximate to the distal end of the channel can have a larger size (e.g., a larger diameter) relative to the openings proximate to the proximal end of the channel. In some embodiments, the sizes of the openings (e.g., their diameters) can progressively increase, or decrease, as a function of increasing distance from the proximal end of the channel.

In some embodiments, a tapered channel can have a width that is smaller at its proximal end relative to its distal end. By way of example, FIG. 3C shows an example of such an embodiment, which includes a plurality of tapered gas delivery channels 28′. Each of the tapered channels 28′ has a width that progressively increases as a function of increasing distance from the distribution channel.

In some embodiments, at least two channels can exhibit different taper angles. Further, in some embodiments, in addition to or instead of one or more of the gas-delivery channels, the input channel and/or the distribution channel can have a tapered shape.

Referring again to FIGS. 1A, 1B, and 1C, the upper and the lower layers 14 and 16 of the sleeve 12 can be formed of any suitable biocompatible material, e.g., any suitable biocompatible polymeric material. By way of example, the layers 14 and 16 can be formed of polyethylene. The thickness of the layers is preferably selected such that the sleeve is sufficiently flexible to conform to the contour of a body part, e.g., a limb, as it is wrapped around that body part. By way of example, the thickness of the upper and the lower layers 14 and 16 can be in a range of about 50 micrometers (microns) to about 250 microns. In some embodiments, the layers 14 and 16 are joined together, e.g., via a heat seal or otherwise, so as to generate air-tight seals along their connecting sections such that a gas introduced into the internal channels of the sleeve can exit the sleeve only through the openings.

As noted above, in some embodiments, the sleeve can include a fastening mechanism, e.g., a medical tape, a hook-and-loop mechanism, which allows fixing the sleeve in place after it has been wrapped around a body part, e.g., a limb. By way of example, FIG. 7B shows a sleeve according to the present teachings, which includes such a fastening mechanism 12b. In other embodiments, the sleeve may lack such a fastening mechanism. In some such embodiments, the sleeve can be wrapped about a limb without fastening the edges of the sleeve together. In some such embodiments, the cast materials disposed on top of the sleeve can ensure that the sleeve will remain wrapped around the body part.

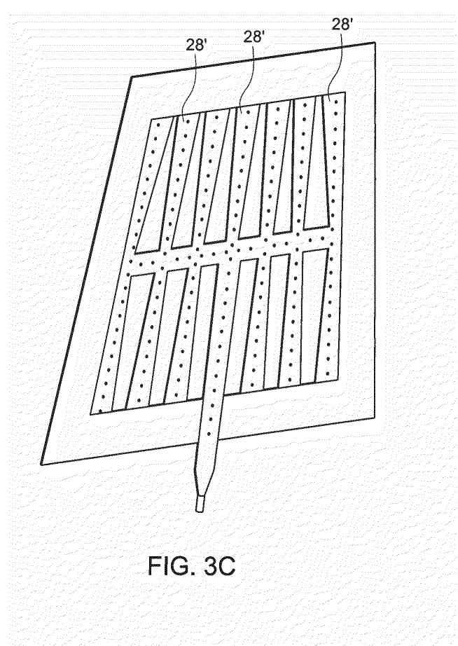

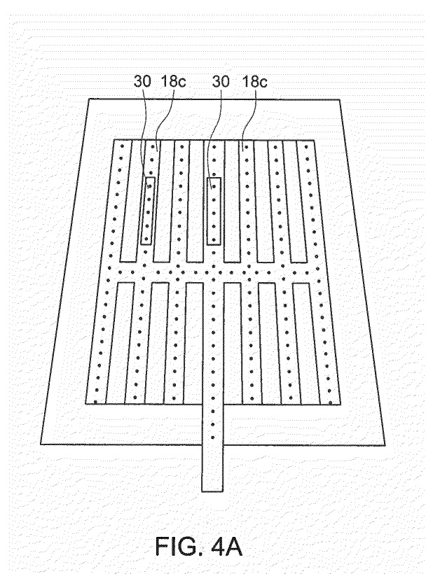

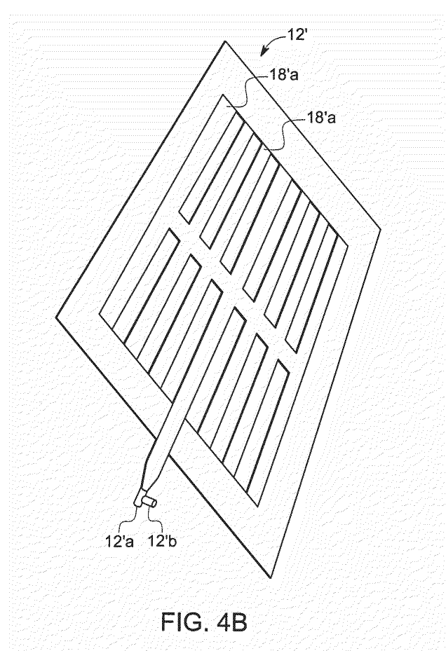

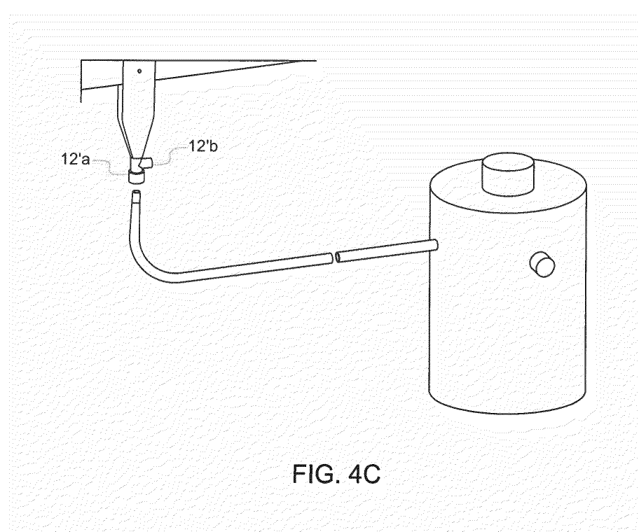

In some embodiments, a porous drug-delivery element, e.g., a sponge soaked with a therapeutic agent, can be disposed in one or more of the delivery channels. By way of example, FIG. 4A schematically depicts an example of such an embodiment, which includes a plurality of porous drug-delivery elements 30, each of which is disposed in one of the gas-delivery channels 18c. By way of example, the drug-delivery elements 30 can be in the form of polyvinyl acetal sponges soaked with a drug. The flow of the gas through the pores of the drug-delivery element can carry the therapeutic agent out of the sleeve onto the skin.

In some embodiments, a sleeve according to the present teachings can include a port coupled to at least one of the channels, e.g., the input channel, to allow the introduction of a drug, e.g., the injection of a drug, into that channel. By way of example, FIGS. 4B and 4C schematically depict a sleeve 12′ according to such an embodiment, which includes a port 12′a for delivery a gas into the sleeve and a port 12′b for introducing a therapeutic agent, e.g., a liquid or a gaseous drug, into the sleeve via an input channel 18′a. In some embodiments, a self-sealing membrane can cover the drug-delivery port such that a needle of a syringe can penetrate through the membrane to deliver a therapeutic agent to the sleeve. The membrane continues to provide a seal once the needle is removed.

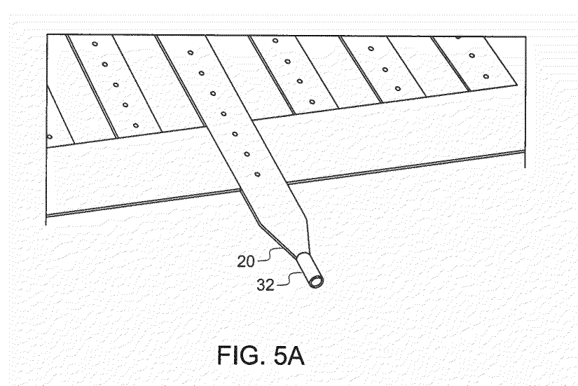

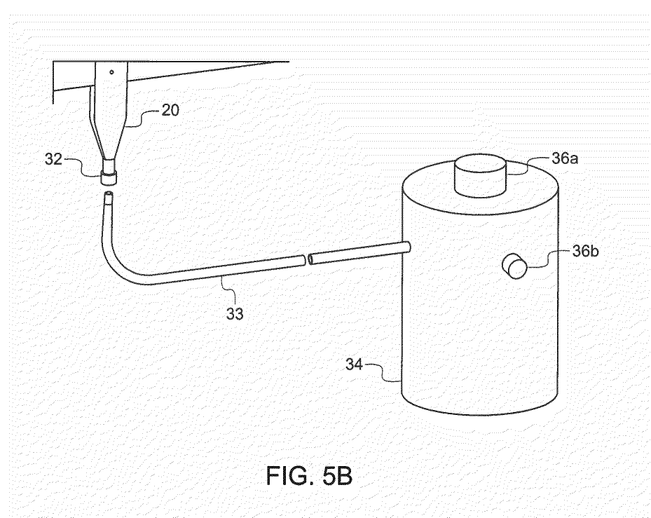

As discussed above, in use, the sleeve can be coupled to an external gas source to receive gas therefrom. For example, with reference to FIGS. 5A and 5B, a gas connector 32, e.g., a quick connect, can be coupled to the opening (inlet) 20 of the sleeve 12 to allow connecting the sleeve to an external source of gas 34. As shown schematically in FIG. 5B, the gas connector 32 can be coupled to a tubing 33 (e.g., a plastic tubing), which can in turn be coupled to the gas container 34, e.g., a canister of compressed gas, for providing gas to the sleeve. In this embodiment, the gas container 34 can include a regulator 36a to control the gas pressure and a trigger mechanism 36b to control the flow rate of the gas exiting the container to enter the sleeve. The use of the regulator advantageously ensures that the gas delivery pressure can be maintained at a desired level, even as the gas volume in the gas container decreases. The trigger mechanism 36b can include a knob that a user can employ to deliver the gas into the sleeve, and hence onto the skin below a cast. For example, the knob can be pressed to initiate the delivery of the gas to the sleeve. By way of example, the user can continue delivering the gas into the sleeve by applying a compressive pressure to the knob, and can release the pressure to discontinue the gas delivery into the sleeve. A variety of gas flow rates into the sleeve can be employed. By of example, the flow rate into the sleeve can be in a range of about ⅓ ft³/min (cubic feet per minute) to about 1 ft³/min (corresponding to about 0.01 to about 0.03 m³/min (cubic meters per minute)). In some embodiments in which a pressurized gas container, e.g., a pressurized CO₂ canister, is employed as the source of gas, the exit of the gas from the pressurized environment of the canister into the sleeve can cause cooling of the gas, which can in turn cool the skin onto which it flows.

A variety of gases can be employed in the practice of the present teachings. Some examples of suitable gases include, without limitation, CO₂, N₂, air, and noble gases, such as argon.

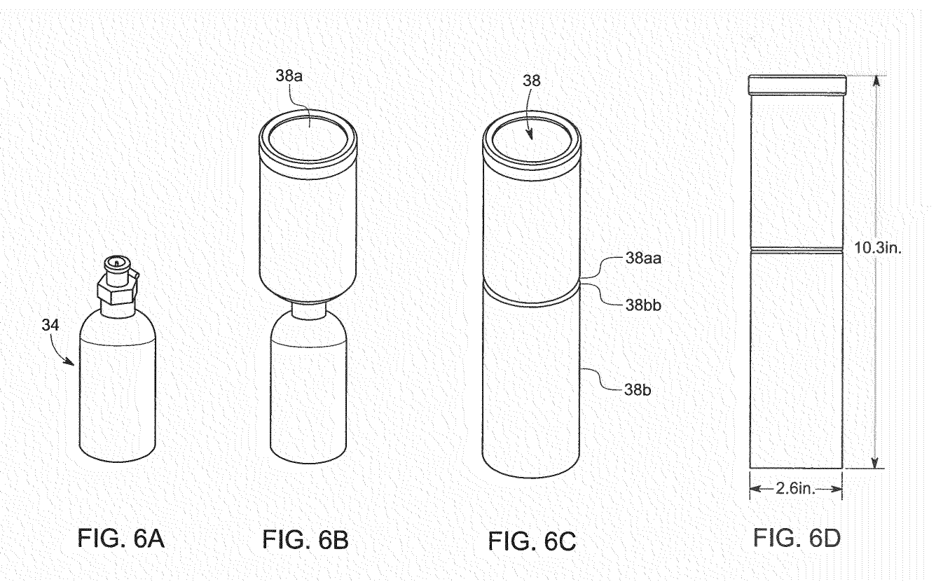

In some embodiments, a container (herein also referred to as a “housing”) can be provided for housing the gas canister. For example, with reference to FIGS. 6A, 6B, 6C, and 6D, in this embodiment, the gas container 34 can be disposed in a cylindrical housing 38, which can facilitate portability of the gas container. The housing 38 includes an upper portion 38a and a lower portion 38b. In this embodiment, the lower portion of the housing has a cylindrical shape and is configured to receive a body portion of the gas container 34 and the upper portion of the housing, which also has a cylindrical shape, is configured to fit over an upper portion of the gas canister, including the nozzle. In this embodiment, upon mounting the upper and lower portions of the housing onto the gas canister, the bottom rim of the upper portion will be flush with the upper rim of the lower portion. In some embodiments, the lower rim (38aa) of the upper portion 38a and the upper rim (38bb) of the lower portion 38b of the housing 38 can be magnetic so that the two portions can be coupled to together via magnetic forces between the upper and the lower portions 38a and 38b.

In some implementations of such an embodiment, the lower cylindrical housing portion can have a height in a range of about 10 inches to about 12 inches (corresponding to about 25.4 cm to about 30.5 cm) and a diameter in a range of about 2.5 inches to about 2.8 inches (corresponding to about 6.4 cm to about 7.1 cm). By way of illustration, FIG. 6D depicts exemplary dimensions for the housing 38.

As shown in FIG. 7A, the housing 38 can include an opening 40 in the upper portion thereof to allow access to the trigger mechanism for delivering gas to the sleeve.

The housing 38 can advantageously facilitate the transport of the gas container by a user, e.g., a patient wearing a sleeve according to the present teachings and a cast. Further, the housing 34 can be formed of a thermally insulated material to provide thermal insulation between a user's hand and the gas container. The housing 34 can be formed of a variety of materials, such as, plastic, e.g., ABS (acrylonitrile butadiene styrene) plastic.

FIG. 7C schematically depicts a sleeve 100 according to another embodiment, which includes a bottom layer 102, and an opposed top layer 104 (the sleeve, and particularly the thickness of the layers, is not drawn to scale for ease of illustration). As in the previous embodiments, the sleeve 100 is configured for mounting to a body part, e.g., a limb, to be placed between the skin and an orthopedic cast. Similar to the previous embodiments, the sleeve is formed of a biocompatible material, e.g., a biocompatible polymer, which is sufficiently flexible to conform to the shape of a body part onto which it is mounted.

As discussed in more detail below, the sleeve can deliver a flow of a gas onto the skin. The bottom layer 102 and the top layer 104 are coupled to one another to form an enclosure therebetween. For example, the perimeters of the top and the bottom layers can be joined together, e.g., via heat sealing or otherwise, to provide the sleeve with an airtight perimeter. Further, a plurality of fasteners 106 can be employed to further couple the top layer to the bottom layer at a plurality of locations. The sleeve 100 includes an inlet 110 for introducing a gas into the enclosure formed between the bottom and the top layers, e.g., in a manner discussed above in connection with the previous embodiments. An external gas source, e.g., a canister of pressurized gas, can supply the gas to the sleeve 100. The bottom layer 102 is formed of a porous material to allow the gas introduced into the enclosure, or at least a portion thereof, to exit the sleeve. In use, the gas exiting the sleeve flows onto the skin below an orthopedic cast, e.g., to provide relief from an itching sensation, and cooling. By way of example, in some embodiments, the porous bottom layer 102 is formed as a porous polyurethane layer.

A combination of the sleeve 12, the gas container 34 and associated fitting and tubing provides an orthopedic system according to the present teachings, which can be used in conjunction with a surgical cast, as discussed above and as further illustrated below.

A variety of manufacturing techniques known in the art can be employed to fabricate a flow sleeve according to the present teachings, e.g., the sleeve 12. By way of example, in some cases, two polymeric layers, e.g., polyethylene layers, can be heat sealed around their perimeters as well other selected sections thereof to form a sleeve having an pattern of internal channels as discussed above. In other embodiments, a molding process can be employed to fabricate a flow sleeve according to the present teachings.

In use, an orthopedic device according to the present teachings can be placed between a subject's skin and an orthopedic cast. The flow of the gas provided by the sleeve onto the skin below the cast can alleviate the itching sensation that typically develops when a patient wears a cast, e.g., to immobilize a broken limb. In many embodiments, the gas flow can cool the skin, and can further remove moisture from the space between the skin and the cast material. This can help alleviate the itching sensation. Further, it can lower the risk of infection.

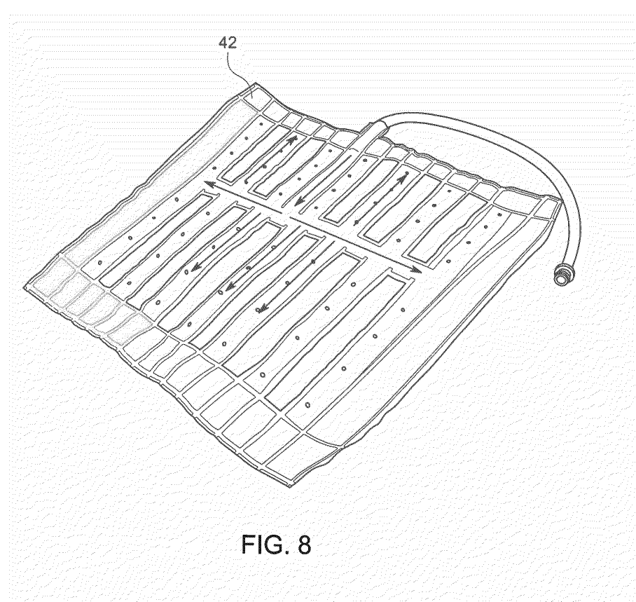

By way of further illustration, FIG. 8 shows a proto-type flow sleeve 42 fabricated in accordance with the present teachings. The arrows are added to the image to indicate the gas flow from an input channel, which is connected via a quick connect to a tube, to receive gas from an external gas source

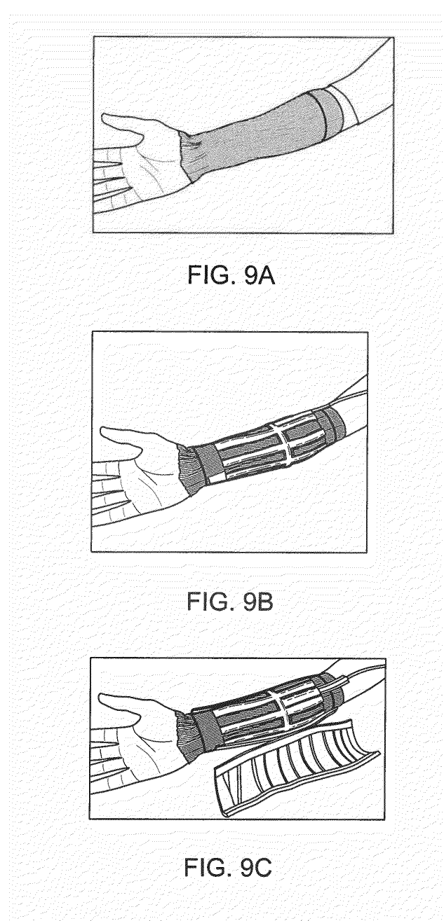

FIGS. 9A, 9B, and 9C show an exemplary way that the sleeve 42 can be mounted to a forearm of a subject to be used with an orthopedic cast. More specifically, as shown in FIG. 9A, initially, a soft, breathable cotton stockinette can be slid onto the arm. Subsequently, the flow sleeve 42 can be wrapped around the stockinette, as shown in FIG. 9B. As shown in FIG. 9C, a casting material can then be applied such that the forearm is casted.

The sleeve can then be coupled to a gas source to provide a flow of a gas, e.g., CO₂, to the sleeve. In some embodiments, the inlet port of the sleeve can protrude a few inches out of the cast to facilitate the connection of the sleeve to a source of gas. As discussed above, the sleeve delivers the gas into a space between the sleeve and the skin so as to provide a gas flow over the skin under the cast. The user, e.g., a patient, can adjust the flow rate of the gas into the sleeve, and consequently the gas flow onto the skin, e.g., by adjusting a regulator coupled to the nozzle of the gas container. In some embodiments, the gas can be delivered to the sleeve via a plurality of gas pulses.

In some embodiments, a flow sleeve according to the present teachings can be formed of a contiguous polymeric layer, rather than two distinct layers joined together as discussed above, to provide an internal enclosure in which a plurality of flow channels are provided. In some such embodiments, a portion of the contiguous layer can be configured for positioning proximate to, or in contact with, the skin upon mounting the sleeve onto a body part and can include a plurality of opening for delivering gas from one or more internal channels of the sleeve onto the skin. The internal channels can be defined, e.g., in a manner discussed above in connection with the previous embodiments. For example, the internal channels can include an input channel having an inlet for receiving gas from a gas source, at least one distribution channel fluidly coupled to the input channel and a plurality of gas-delivery channels fluidly coupled to the distribution channel. A plurality of openings can be provided on the portion of the sleeve adapted to be proximate to, in contact with the skin, so as to deliver a flow of gas from the sleeve onto a patient's skin.

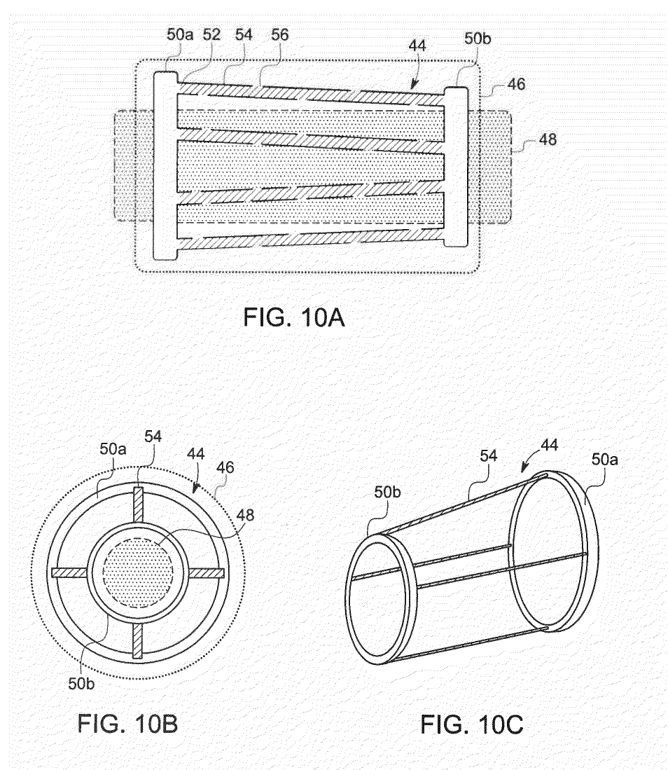

With reference to FIGS. 10A, 10B, and 10C, an orthopedic device according to another embodiment of the present teachings may include a sleeve 44 (herein also referred to as a “tube sleeve”), which is configured to be mounted on the exterior of a subject's body part 48, e.g., a limb, between the skin and an orthopedic cast 46. In this embodiment, the sleeve 44 may include a first cuff 50a and a second cuff 50b at opposing ends thereof. Although FIGS. 10A, 10B, and 10C depict two cuffs, 50a and 50b, embodiments are not so limited as the sleeve 44 may include any number of cuffs 50a, 50b that may operate according to some embodiments described herein.

The first cuff 50a and the second cuff 50b may be substantially circular and may be configured to encircle a portion of the subject's body part 48. For example, for an orthopedic cast 46 configured to cover a portion of a subject's forearm, the first cuff 50a may be configured to encircle a portion of the upper forearm and the second cuff 50b may be configured to encircle a portion of the lower forearm, such as a portion adjacent to the wrist. In another example, for an orthopedic cast 46 configured to cover a portion of a subject's calf, the first cuff 50a may be configured to encircle a portion of the upper calf and the second cuff 50b may be configured to encircle a portion of the lower calf, such as a portion adjacent to the ankle

The first cuff 50a and the second cuff 50b may be hollow and, as such, may be configured as substantially circular channels having at least one opening 52, for example, on facing surfaces thereof. A plurality of channels 54 may be arranged substantially parallel to one another, and the subject's body part 48, and substantially orthogonal to the first cuff 50a and the second cuff 50b. The plurality of channels 54 may be configured to connect an opening 52 on the first cuff 50a with a corresponding opening 52 on the second cuff, thereby connecting the first cuff 50a and the second cuff 50b in fluid communication. In some implementations of such an embodiment, the sleeve 44 may include about 3 to 10 channels 54.

The sleeve 44 may be coupled to a gas source to provide a flow of a gas, e.g., CO₂, to the sleeve 44. In some embodiments, the sleeve 44 may include an inlet port that may protrude a few inches out of the orthopedic cast 46 to facilitate the connection of the sleeve 44 to a source of gas. A plurality of perforations (openings) 56 may be disposed in each of the plurality of channels 54 to allow passage of the gas from the plurality of channels 54 onto the skin of the subject's body part 48. In some embodiments, the plurality of perforations 56 may be disposed on the surface of the plurality of channels 54 facing the skin of the subject's body part 48. In some embodiments, the perforations 56 may have a diameter of about 0.25 centimeters to about 3 centimeters.

As discussed above, the sleeve 44 delivers the gas into a space between the sleeve 44 and the skin so as to provide a gas flow over the skin under the cast. The user, e.g., a patient, can adjust the flow rate of the gas into the sleeve, and consequently the gas flow on the skin, by adjusting a regulator coupled to the nozzle of the gas container. In some embodiments, the gas can be delivered to the sleeve 44 via a plurality of gas pulses.

In some embodiments, the first cuff 50a, the second cuff 50b, and/or the plurality of channels 54 may be formed from a flexible material. In some embodiments, the first cuff 50a, the second cuff 50b, and/or the plurality of channels may be formed from a flexible material that is sufficiently firm to maintain a shape to ensure an even flow of gas through the sleeve 44. In some embodiments, the flexible material may include a biocompatible polymer. In some embodiments, the biocompatible polymer may include a polyethylene polymer, such as a low-density polyethylene polymer (LDPPE). A non-limiting example of a LDPPE is Tygon® tubing made by the Saint-Gobain S.A. of La Defense, France, such as ⅛″ inner diameter Tygon® tubing. In some embodiments, the first cuff 50a, the second cuff 50b, and/or the plurality of channels may have an inner diameter of about 1 centimeter, about 2 centimeters, about 3 centimeters, about 4 centimeters, about 5 centimeters, and any value or range between any two of these values (including endpoints).

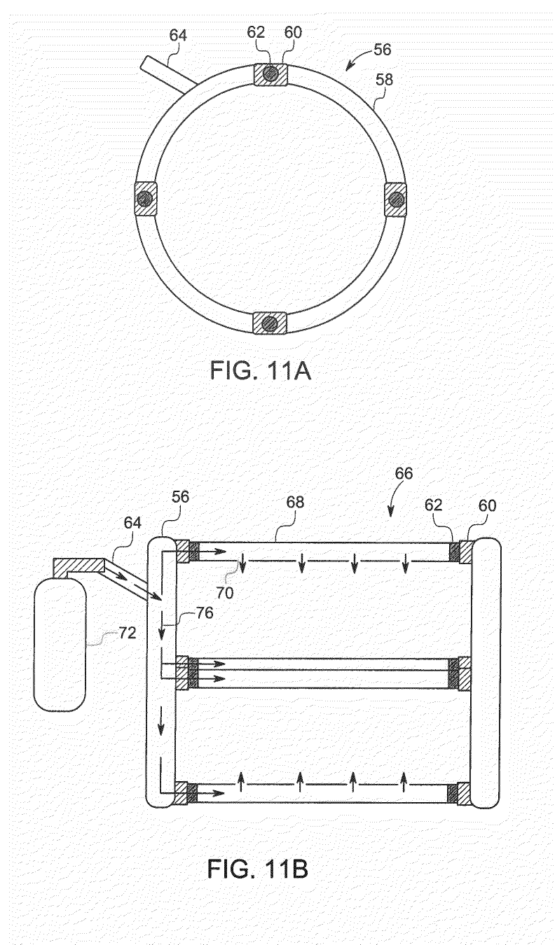

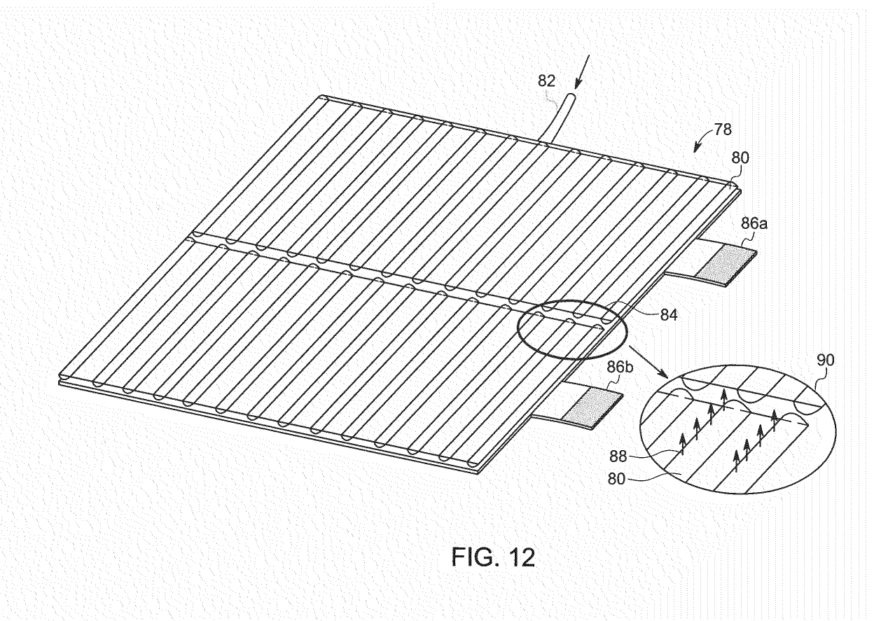

With reference to FIGS. 11A and 11B, a sleeve 66 may include cuffs 56 formed from a plurality of channel sections 58. In some embodiments, the channel sections 58 may be formed from flexible tubing, such as Tygon® tubing. The channel sections 58 may be connected via connectors 60 having an opening 62 configured to receive a channel 68. In some embodiments, the connectors 60 may include tee connectors. A gas source 72 may be connected to the sleeve 66 via a port 64, such as a barbed fitting, disposed in or in fluid communication with a cuff 56. A gas flow 76 may flow from the gas source 72 and through the components of the sleeve 66. The channels 68 may include perforations 70 configured to allow the gas flow 76 to flow out of the channels and, for example, onto the skin of a subject's body part. In some embodiments, the connection points between the connectors 60 and the cuffs 56 and/or the openings 62 and the channels 68 may be covered with an adhesive to facilitate a permanent or semi-permanent connection therebetween. In some embodiments, the adhesive may include an epoxy.

In some embodiments, at least a portion of the sleeve 66 may be covered by a material. In some embodiments, the material may include a fabric that is soft, stretchable and/or non-allergenic. In some embodiments, the material may be configured to wick moisture away from the skin of a subject's body part. In some embodiments, the material may be or may be similar to Dri-Fit® material manufactured by Nike® of Beaverton, Oreg., United States.

With respect to FIG. 12, a sleeve 78 may be formed as a bladder (also referred to as a “bladder sleeve” or “bladder” herein) configured to encircle a portion of a limb, such as a portion covered by and/or adjacent to a cast. In some embodiments, the sleeve (or bladder) 78 may be configured to be wrapped around the limb. In some embodiments, when wrapped around a limb, each end of the sleeve 78 may be secured by one or more securing structures 86a, 86b, for example, adhesive patches. The sleeve 78 may include a plurality of channels 80 disposed therein. In some embodiments, the plurality of channels 80 may be parallel or substantially parallel with respect to each other.

In some embodiments, the sleeve 78 may include an inlet port 82 that may protrude a few inches out of the orthopedic cast to facilitate the connection of the sleeve 78 to a source of gas. The inlet port 82 may be in fluid communication with at least a portion of the plurality of channels 80. In this manner, gas entering the sleeve 78 through the inlet port 82 may flow into at least a portion of the plurality of channels 80. A plurality of perforations (openings) 88, as seen in detail 90 of area 84 of the sleeve 78, may be disposed in each of the plurality of channels 80. The plurality of perforations 88 may be configured to allow passage of the gas from the plurality of channels 80, for example, and onto the skin of the subject's body part and/or an inside surface of the cast. In some embodiments, the plurality of perforations 88 may be disposed on the surface of the plurality of channels 80 facing the skin of the subject's body part.

In some embodiments, the sleeve 78 may be formed from a flexible material, including, without limitation, a polymer material. In some embodiments, the flexible material may include a biocompatible material. In some embodiments, the flexible material may include polyethylene and derivations thereof. In some embodiments, the sleeve may have a thickness, when filled with gas or substantially filled with gas of about 25 microns, about 50 microns, about 75 microns, about 100 microns, about 150 microns, about 200 microns, and about 300 microns.

In some embodiments, the sleeve 78 may be formed from two polymer sheets, such as polyethylene. The plurality of channels may be formed using an impulse heat sealer, such as a heat sealing press, configured to heat up the two polymer layers and to fuse them into one sleeve 78 having a plurality of channels 80 arranged therein. The sleeve 78 may be arranged within an air-permeable material that may be in direct contact with the skin. In some embodiments, the air-permeable material may be formed as a stockinette, such as a soft, breathable cotton stockinette. In some embodiments, the air-permeable material may be in the form of a compression sleeve, for example, formed from about 90% polyester and about 10% spandex blend.

Those having ordinary skill in the art will appreciate that various modification can be made to the above embodiments without departing from the scope of the invention. For example, the features of one embodiment can be incorporated in another embodiment.

1. An orthopedic device for use with an orthopedic cast, comprising:
a sleeve having opposed top and bottom layers and adapted for mounting onto a subject's body part such that the bottom layer is disposed proximate to, or in contact, with the skin,
said opposed top and bottom layers being connected to one another so as to form an input channel, a distribution channel and at least one gas-delivery channel in a space therebetween,
said input channel comprises an opening at a proximal end thereof for receiving gas from an external source, said input channel being fluidly coupled to said distribution channel at a distal end thereof to deliver said received gas to said distribution channel,
said distribution channel being configured to deliver at least a portion of said gas to each of said plurality of gas-delivery channels,
a plurality of openings disposed in said bottom layer to allow egress of the gas from at least one of said channels out of the sleeve.

2. The orthopedic device of claim 1, wherein said distribution channel is substantially orthogonal to said input channel.

3. The orthopedic device of claim 2, wherein said distribution channel is substantially orthogonal to said at least one gas-delivery channel.

4. The orthopedic device of claim 1, wherein each of said channels includes a top wall formed by a portion of said top layer and a bottom wall formed by a portion of said bottom layer.

5. The orthopedic device of claim 4, wherein said openings are disposed along a bottom wall of said gas-delivery channel.

6. The orthopedic device of claim 5, wherein said plurality of openings are separated uniformly from one another.

7. The orthopedic device of claim 5, wherein said openings are randomly disposed relative to one another.

8. The orthopedic device of claim 1, wherein said at least one gas-delivery channel comprises a plurality of gas-delivery channels disposed substantially parallel to one another.

9. The orthopedic device of claim 8, wherein said gas-delivery channels are substantially perpendicular to said distribution channel.

10. The orthopedic device of claim 1, wherein said at least one gas-delivery channel extends from a proximal end in communication with said distribution channel to a distal end.

11. The orthopedic device of claim 10, wherein said at least one gas-delivery channel has a tapered shape.

12. The orthopedic device of claim 11, wherein said tapered shape of said gas-delivery channel is characterized by a width at said distal end thereof.

13. The orthopedic device of claim 11, wherein said tapered shape is characterized by a taper angle in a range of about 10 degrees to about 30 degrees.

14. The orthopedic device of claim 1, further comprising a porous drug-delivery element disposed in said at least one gas-delivery channel.

15. The orthopedic device of claim 11, wherein said porous drug-delivery element comprises a vinyl polymer.

16. The orthopedic device of claim 12, wherein said vinyl polymer comprises vinyl acetate.

17. The orthopedic device of claim 1, wherein said openings have any of a circular, an elliptical, a square shape, or an irregular shape.

18. The orthopedic device of claim 1, wherein said openings have a circular shape with a diameter in a range of about 0.8 mm to about 1.6 mm.

19. The orthopedic device of claim 1, wherein said of said layers has a thickness in a range of about 50 microns to about 250 microns.

20. The orthopedic device of claim 1, wherein each of said layers is formed of a biocompatible material.

21. The orthopedic device of claim 20, wherein said biocompatible material comprises polyethylene.

22. The orthopedic device of claim 1, wherein said bottom layer has a corrugated outer surface.

23. The orthopedic device of claim 22, wherein said corrugated surface includes a plurality of grooves providing a plurality of cavities between the skin and the sleeve when the sleeve is mounted onto a body part, where said cavities facilitate contact between the gas exiting the sleeve and the skin.

24. The orthopedic device of claim 1, wherein said external gas source comprises a container of compressed gas.

25. The orthopedic device of claim 24, wherein said gas comprises any of CO₂, N2, air and argon.

26. The orthopedic device of claim 24, wherein said container comprises a regulator for adjusting flow of the gas exiting the container.

27. The orthopedic device of claim 26, further comprising a fitting coupled to said opening of the input channel.

28. The orthopedic device of claim 27, further comprising a conduit extending from said container to said fitting for delivering the gas to said input channel of the sleeve.

29.-60. (canceled)
